(19) United States
(12) Patent Application Publication
(10) Pub. No.: US 2008/0015482 A1
(43) Pub. Date: Jan. 17, 2008

(54) Resilient double pad hemostasis devices

(76) Inventors: David S. Utterberg, Seattle, WA (US); William J. Schnell, Libertyville, IL (US); David Bell, Grayslake, IL (US)

Correspondence Address: Garrettson Ellis;Seyfarth Shaw, 42nd Floor, 55 E Monroe Street, Chicago, IL, 60603, US

(21) Appl. No.: 11/485,910

(22) Filed: Jul. 13, 2006

(51) Int. Cl. A61F 13/00

(52) U.S. Cl. 602/48

(57) ABSTRACT

A soft hemostasis pad of elastomeric material having a hardness of no more than about 50 Shore 00 and an upper surface of raised profile, such as a dome. The pad may be connected by a hinge with another, generally flat-bottom pad of similar elastomeric material and hardness of no more than about 50 Shore 00. The pad may rest on the skin and be penetrated by a needle or other cannula. The pad with the raised profile may then be placed over on top of the needle, for improved hemostasis and sealing when the cannula is removed and before, facilitated by the application of gentle pressure, such as by tape that holds the pads in place.

### BACKGROUND OF THE INVENTION

In U.S. Patent Application Publication 2006/0079823 A1, entitled Injection and Hemostasis Site, soft, hemostasis pads are disclosed, made of the elastomeric material having a hardness of no more than about durometer 50 Shore 00. This is a very soft elastomer, providing hemostasis after the withdrawal of needles and catheters from the skin, for example the rather large fistula needles used in hemodialysis. Used as a substitute for gauze, they may create hemostasis of a fistula or the like with much less loss of blood, and with much less pressure required to halt the blood flow, when compared with gauze. It is believed that this reduction in pressure which can be provided by such resilient elastomeric hemostasis devices reduces damage to the fistula, or a graft, or the like for longer life thereof.

Also, Master Seal™ hemostasis gel pads sold by Medisystems corporation, utilize dome-shaped, solid, optically clear, flat-bottomed soft elastomer pads for hemostasis upon the withdrawal of medical sharp needles from the vascular system.

Also, Harren et al., U.S. Pat. No. 6,007,562 and Becher, U.S. Pat. No. 5,236,421 show multiple layer puncture closure systems through which a cannula may penetrate to reach the skin.

By this invention, improvements are provided in the field of hemostasis, for catheters and needle systems both during implantation thereof and after withdrawal, reducing both blood seepage while a percutaneous catheter or sharp needle is emplaced in the patient, and providing sealing hemostasis after withdrawal thereof.

### DESCRIPTION OF THE INVENTION

By this invention, a method comprises: applying soft, first pad to the skin of a patient. The first pad consists essentially of an elastomer, solid or with an internally porous structure, having a hardness of no more than about Durometer 50 Shore 00. A medical cannula is passed through the pad into tissue of the patient in a manner described in the cited Patent Application Publication No. US2006/0079823 A1, the disclosures of which are incorporated by reference herein. Then, a second, soft pad is placed to overlie the first, soft pad and medical cannula, the second, soft pad having a raised profile upper wall and being of an elastomeric material having a hardness of no more than about 50 Shore 00, for example being of dome shape with a generally flat lower surface. The two pads may be hinged together. The second, soft pad is secured in place on the first pad with downward pressure, typically a gentle applied downward pressure of less than that normally used in typical cotton swab hemostasis, to avoid blood vessel collapse and to achieve improved hemostasis, reduced blood loss, and the other advantages of this invention as described in the cited patent application publication. This may be accomplished by taping the pads to the skin with tape extending over the typically raised profile upper wall surface.

Then, typically, the cannula may be withdrawn from the patient, without removing the first and second soft pads from their positions, for improved suppression of bleeding, both before the cannula is removed and afterward. The cannula may comprise a sharp or dull hollow needle, a catheter, a trocar, or the like.

The assembly of pads used in this invention may all abut together to prevent formation of a substantial blood chamber therein. Also, the respective pads each may comprise a single layer of material.

The first, soft pad, in some embodiments, may be clear, soft, and thin enough, typically on the order of 1 to 5 millimeters, so that the cannulation site of a patient may be palpitated through the already in-place first pad prior to cannulation, in order to find an underlying vessel or medical device by touch, so as to maintain the sterility of the patient's intended cannulation site, and to seal it. If desired, the elastomeric pad may be coated with disinfectant, or it may be applied to pre-disinfected skin, before cannulation, or applied after withdrawal of the cannula, to prevent bleeding without necessarily continuing applied, strong, external pressure.

Upon cannulation, an advancing needle or catheter can pass through the first elastomeric pad and then into the skin of the patient, typically into the vascular system of the patient. Upon withdrawal of the cannula, the elastomeric first pad of the specified softness exhibits the surprising property of resealing without a compressive housing, and, because of its substantially non-absorbant lower surface, provides significant suppression of bleeding, with a consequent decrease of hemostasis time in a patient, with significantly less need for compression to facilitate the hemostasis.

The hemostasis pads described herein may be made of a substantially transparent, pore-free material, although non transparent, or closed-cell materials may be used, if desired, having a smooth, typically pore free, substantially non-absorbant lower surface. Harder elastomer materials may be softened to a desired softness for use herein by the inclusion of gas or air-containing foam cells, if desired.

In some embodiments, the hardness may be no more than about 30 or 35 Shore 00. For example, a currently favored material for manufacturing the pad is made by Gel Concepts, LLC of Whippany, N.J., being a soft, transparent, oil plasticized elastomer having a Shore 00 hardness of about 10.

Another suitable material is Versaflex® CL2003X thermoplastic rubber compound, which is a proprietary, oil-plasticized styrene block copolymer elastomer, manufactured by GLS Corp. of McHenry, Ill. This is another transparent material with a Shore 00 hardness of about 29, and having other physical parameters as described in the above-cited Patent Application Publication No. 2006/0079823.

If desired, pads in accordance with this invention may carry a medicament, particularly to be present on a typically generally flat, solid and nonporous bottom of the above-described first pad. Such a medicament may be an antiseptic of any desired kind. Particularly, commercially available silver nanoparticles may be available, to be incorporated in effective concentration, such as 0.001 to 0.1 wt. percent, into the formulation of the material comprising the first pad, or both pads, but such nanoparticles do not strongly reduce the transparency of the pad or pads, so that the pads may have antiseptic characteristics and remain transparent. Any other desired antiseptic may also be included, for example, alcohol such as ethanol or isopropyl, and other known antiseptics, either applied to the lower surface of the first pad or incorporated in the pad material itself for similar effect.

It is also of advantage for the elastomeric material of the first pad or both pads to contain a liquid plasticizer such as mineral oil, so that the material of the pad does not significantly adhere to a scab formed by bleeding under the solid bottom as the pad rests on the skin of the patient. Thus, the first pad is easily removed without opening the scab, after the need to provide hemostasis is no longer present.

The pads may comprise, as stated, an elastomer having a hardness of no more than about 50 Shore 00, and preferably no more than 30 or 35 Shore 00. The second pad may have a dome shape or other raised profile upper surface, with a bottom that presses the first pad, so that when gentle pressure is exerted on the top of the dome, it is efficiently transmitted to the bottom, providing a low pressure, less than was generally provided with cotton swab hemostasis, but because of the softness of the pad, the elastomer material obstructs pathways surrounding the injury site (caused by a catheter, needle, or a skin injury coming from any source) preventing the flow of blood away from the injury site. Typically, the material of at least the first pad is of approximately the softness of skin, which greatly improves the sealing ability of its lower surface to the skin adjacent to it, when compared with other materials.

Although in some embodiments a generally flat bottom on the first pad will best conform to the patient's skin at the cannulation or percutaneous entry site, a slightly concave or convex bottom can in some circumstances better conform to the curving topography on a cannulation or percutaneous entry site on an arm, leg, or the like. Such a fitting concave or convex shape may be deemed “generally flat”.

Other raised profiles that the second pad of this invention may exhibit may include other types of convex, upper wall. For example, a convex, upper wall may curve in one dimension, contrary to a dome which curves in two dimensions, so that an upper wall is of a shape of an inverted trough, having substantially similar cross section along its length. Particularly, if the second pad is not attached to the first pad, the second pad can be extruded through a die of D-shaped cross section, for efficient manufacture.

The first pad may comprise a disk, rectangle, or the like of essentially constant thickness, and thus is easily manufactured by being stamped out of extruded sheeting of the desired elastomeric material, particularly if unattached to the second pad.

Further, as described above, the pads may be transparent, and an antiseptic, as before, may be present on the lower surface of the first pad. Optionally the antiseptic is an integral part of the formulation of the entire pad. Silver nanoparticles, as stated before, may be used.

If desired, the first and second pads may be integrally molded from a transparent, typically substantially pore-free material with an integral plastic hinge connecting them, permitting them to fold together, where an upper face surface of the first pad engages the lower surface of the second pad.

The pads may be taped to the skin in a conventional manner over the cannula, i.e., a hollow medical needle or a catheter, to gently retain the pads in place while the cannula resides through the skin. Both before after the cannula is removed, the pads serve as a hemostasis device. The pads may be held against the skin with gentle pressure, typically less pressure than is applied with gauze, for improved hemostasis, which has a particular advantage of reducing pressure injury to artificial blood vessels such as a fistula, a graft, or the like, as described in Patent Publication 2006/0079823.

### DESCRIPTION OF THE DRAWINGS

Referring to the drawings, FIG. 1 is a perspective view of the connected first and second pads of this invention, shown to be lying on the skin of a patient in open position.

FIG. 2 is a perspective view illustrating how a fistula, vein, or other blood vessel of a patient can be palpitated through the first pad of the device of this invention to locate the blood vessel.

FIG. 3 is a side view showing how a medical cannula can penetrate through the first pad of the device as shown in FIGS. 1-2, and may also penetrate through tissue of the patient, to reach a blood vessel underneath the skin.

FIG. 4 shows how the second pad may then be folded over after penetration of the cannula, to cover the cannula entry site and portions of the cannula.

FIG. 5 is a perspective view showing the configuration of FIG. 4, in reversed direction, after the cannula and device of this invention are taped together and onto the skin of the patient.

### DESCRIPTION OF SPECIFIC EMBODIMENTS

Referring to the drawings, the hemostasis pad device 10 of this invention may comprise a first pad 12 and a second pad 14, connected together by a hinge 16 so that second pad 14 may be placed on top of first pad 12 by folding. This assembly of pads may be integrally molded together as a single, unitary item, if desired or, alternatively, separate first and second pads may be provided and used.

Each of pads 12, 14 are made, in this embodiment, of an elastomeric material having a Shore 00 durometer hardness on the order of 10, for example being a soft, transparent, oil plasticized elastomer such as the material made by Gel Concepts LLC, of Whippany, N.J., or other materials as described in the previously cited patent application publication.

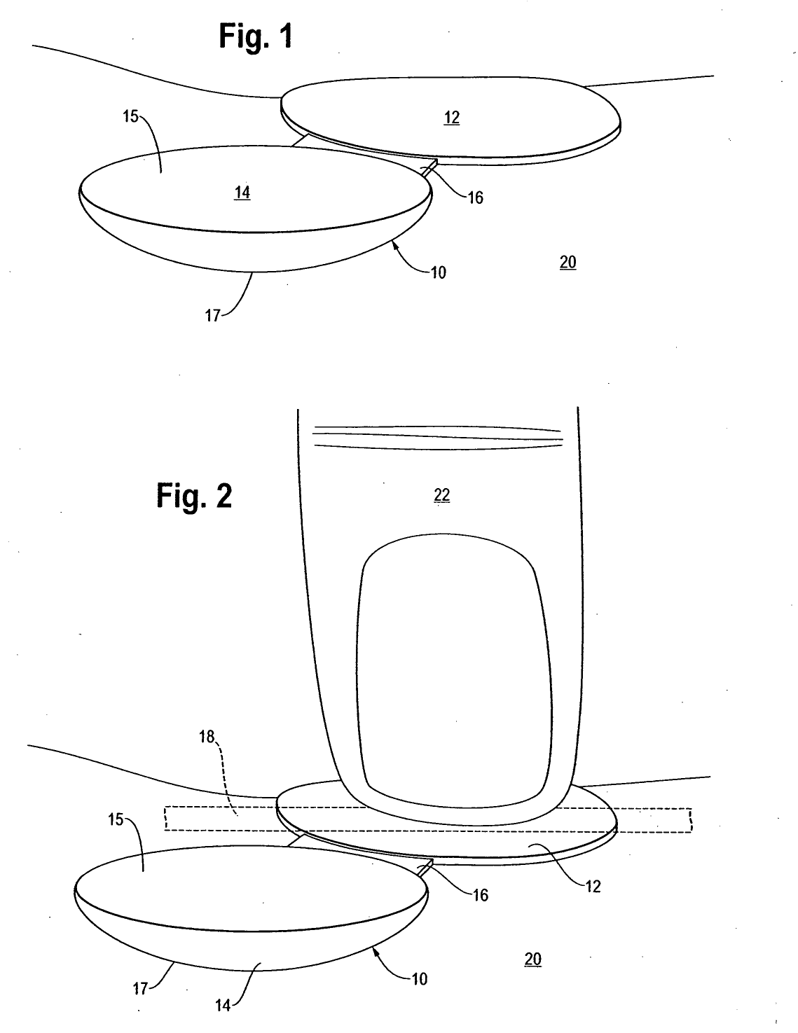

First, soft pad 12 has a generally flat top and bottom, and a thickness on the order of 0.1 to 0.5 cm, so that it is possible to palpitate a blood vessel (FIG. 2) such as a fistula or a vein 18 under the skin 20 of the patient with a finger 22. It can be seen that FIG. 2 is significantly enlarged, when comparing the size of finger 22 to a normal finger. The diameter of each of first and second circular pads 12, 14 may be similar, in some embodiments, and may be on the order of 1-5 centimeters, for example. The pads may also be oval, rectangular, triangular, or the like.

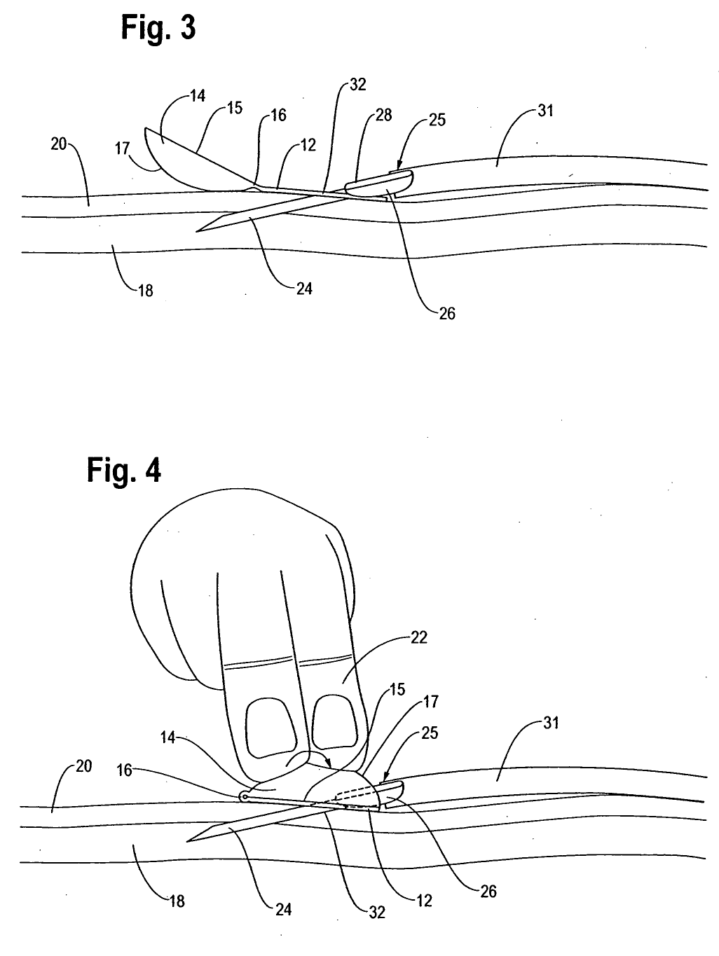

As shown in FIGS. 1-3, both pads 12, 14 of the pad device 10 are shown to be lying on the patient's skin 20.

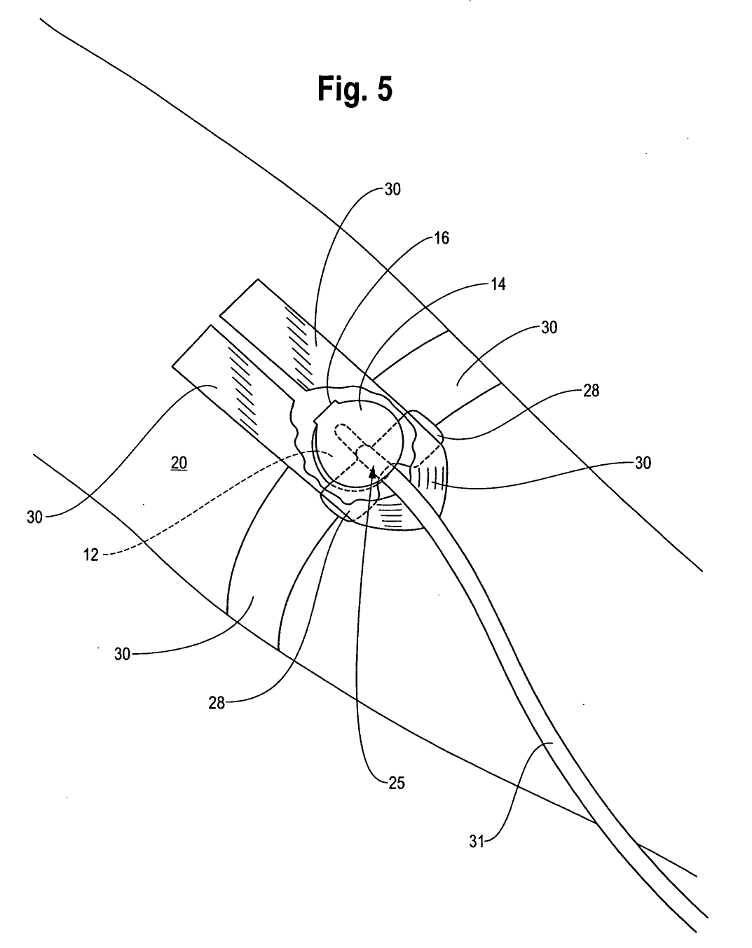

Second pad 14 is inverted from its normal position of use in FIG. 1, but is folded over as in FIG. 4, for use, so that its lower surface, 15, in use, is generally flat, and it has a raised-profile upper surface 17, which surfaces are inverted in FIGS. 1-3. Raised profile upper surface 17 of second pad 14 may be of a dome shape, but, as previously described, may be of another, desired, typically raised-profile shape, one such alternate shape being as shown in FIG. 5 of the previously cited U.S. patent application publication. Surface 17 may alternatively comprise a cubic structure or the like, to provide a raised profile where, typically, central portions of surface 17 are higher than peripheral portions of surface 17. The purpose of the raised profile is to facilitate the distribution of gentle, downward pressure of second pad 14 onto the surface of first pad 12 when folded on it as in FIG. 4, to achieve the advantages that can be achieved with the gentle pressure, applied to effectively stop bleedings while being gentle enough to not close off blood vessel 18, this being described in the previously cited patent application publication.

As shown in FIG. 2, blood vessel 18 is palpitated to determine its location within skin 20 by finger 22 in a conventional manner. Because of the thinness and softness of first pad 12, it is possible to palpitate blood vessels through the pad, as shown.

Then, when blood vessel 18 is so located by palpitation through first pad 12, a sharpened cannula such as a conventional winged needle set 25, having a conventional needle 24, is placed through pad 12 and skin 20 into blood vessel 18. Needle set 26 may also comprise a conventional hub 26, and wings 28, attached in conventional manner to flexible set tubing 31 for example, so that needle set 25 and tubing 31 may comprise a conventional fistula set. Needle 24 is conventionally inserted into blood vessel 18, which has been located by palpitation through pad 12, so that the needle hole 32 through skin 20 is located underneath first pad 12, and the needle extends through first pad 12.

Then, second pad 14 is folded about hinge 16 to overlie pad 12 and needle hole 32 in the skin, also overlying at least a portion of the needle 24 and hub 26, as shown in FIG. 4. Because of the high softness of second pad 14, its lower, flat surface 15 flexes to receive and accommodate for the presence of needle 24 and hub 26, to provide a structure that overlies needle hole 32, providing sealing for blood that seeps through needle hole 32 and the corresponding needle hole in first pad 12. Also, first pad 12, itself, by its soft nature and non-absorbent lower surface, as described in the cited patent application publication, provides sealing and prevention of the leakage of blood from skin hole 32.

Then, as shown in FIG. 5, winged needle assembly 25 may be secured to the skin 20 of the patient by strips of tape 30, conventionally applied in a desired, typically conventional pattern to retain needle assembly 25 and the folded pads 12, 14 of this invention in its desired position for the desired duration. This may be a period of hours, for example in the event of a conventional hemodialysis treatment, or even days in other circumstances, such as with an i.v. catheter. Bleeding is suppressed by the presence of the pad device of this invention.

Then, when needle assembly 25 is desired to be removed, it may be simply done by removing tape portions that cover said needle assembly, and withdrawing needle 24, without removal of the folded pads 14, 16. Because of the high softness and resilience of pads 12, 14, they simply close up the space that is vacated by needle hub 26 and needle 24, spontaneously providing an added seal against bleeding. Remaining tape portions that hold folded pads 14, 16 onto the skin may be reinforced with added tape at this point, if desired, for better retention.

The tape portions 30 that pass over the raised profile of upper surface 17 of pad 14 may convey downward pressure to central portions of pad 12 surrounding needle hole 32, to provide a gentle sealing pressure, typically of less pressure than was conventionally used to stop bleeding previously in the prior art, for improved maintenance of blood vessels, particularly fistulas and the like.

In some embodiments, the pads 14, 16 are both transparent and substantially pore free. Also, because of the presence of liquid oil plasticizer in preferred formulations that make the respective pads 14, 16, first pad 12 does not tend to adhere to a scab formed by bleeding under the pad as it rests on the patient, so that it can be more easily removed without damaging the scab.

The above has been offered for illustrative purposes only, and is not intended to limit the scope of the invention of this application, which is as defined in the claims below.

That which is claimed is:

1. The method which comprises: applying a first, soft pad to the skin of a patient, said pad consisting essentially of an elastomer having a hardness of no more than about durometer 50 Shore 00; passing a medical cannula through the pad into the tissue of the patient; overlying the first, soft pad and medical needle with a second, soft pad of elastomeric material having a hardness of no more than about 50 Shore 00, said second pad having a raised-profile upper surface and a generally flat bottom; and
securing the second, soft pad in place on said first, soft pad and said medical needle cannula with pressure against the skin.

2. The method of claim 1 comprising the step of withdrawing the cannula from the patient without removing the first and second, soft pads from their positions, whereby bleeding is suppressed.

3. The method of claim 1 in which the first and second, soft pads are peripherally connected by a hinge.

4. The method of claim 1 in which said first and second, soft pads are integrally formed from a transparent, substantially pore-free material.

5. The method of claim 1 in which the hardness of each of said first and second soft pads is no more than about durometer 35 Shore 00.

6. The method of claim 1 in which the first pad is thin enough to allow palpitation of a blood vessel of the patient through the pad, and including the steps of palpitating the blood vessel prior to passing the medical cannula through the first pad and into the blood vessel.

7. A first, soft, hemostasis pad of elastomeric material having a hardness of no more than about 50 Shore 00, said first pad having a generally flat and non-absorbent lower surface and a second, soft pad peripherally connected by a hinge to said first, soft pad, said second, soft pad comprising an elastomeric material having a hardness of no more than about durometer 50 Shore 00, said second soft pad having a raised profile upper surface and a generally flat, lower surface, said second, soft pad being capable of folding by said hinge to abut the flat, solid bottom of the second, soft pad against a major face of said first, soft pad.

8. The first and second soft pads of claim 7 in which each pad has a hardness of no more than about durometer 35 Shore 00.

9. The first and second soft pads of claim 7 in which said first and second pads are integrally molded.

10. The first and second soft pads of claim 9 in which the second, soft pad has a dome-shaped upper surface.

11. The first and second soft pads of claim 7 in which the elastomeric material of at least said first soft pad contains a liquid plasticizer, whereby said first, soft pad does not significantly adhere to a scab formed by bleeding under the first soft pad as the first, soft pad rests on the skin of a patient.

12. The first and second soft pads of claim 7 in which said raised profile upper surface is a dome.

13. The first and second soft pads of claim 12 in which said first and second pads are integrally molded from a transparent, substantially pore-free material.

14. The first and second soft pads of claim 13 in which the elastomeric material of at least said first soft pad contains a liquid plasticizer, whereby said first soft pad does not significantly adhere to a scab formed by bleeding under the first soft pad as the first soft pad rests on the skin of a patient.

15. The first and second soft pads of claim 14 in which of each of said soft pads has a hardness of no more than about 35 Shore 00.

16. The first and second pads of claim 7 which are of porous construction, except for at least said first pad's lower surface, which is non-absorbent.

17. The method which comprises:
applying a first, soft pad to the skin of a patient, said pad consisting of essentially of elastomer having a hardness of no more than about durometer 50 Shore 00;
passing a medical needle through the first pad into the tissue of the patient;
overlying the first, soft pad with medical cannula with a second, soft pad of elastomeric material having a hardness of no more than about 50 Shore 00, said overlying taking place by folding said second, soft pad about a hinge connected to the first, soft pad, to place said second, soft pad on top of said first, soft pad and needle, said second soft pad having a raised profile upper surface and a generally flat bottom;
securing the second, soft pad in place on said first, soft pad and said medical needle with pressure against the skin;
and thereafter withdrawing the cannula from the patient without removing the first and second, soft pads from their positions, whereby bleeding is suppressed.

18. The method of claim 17 in which said first and second soft pads are integrally formed from a transparent, substantially pore free material.

19. The method of claim 18 in which the hardness of each of said first and second soft pads is no more than about 35 Shore 00.

20. The method of claim 17 in which at least said first, soft pad contains a liquid plasticizer, whereby the first, soft pad does not significantly adhere to a scab formed by bleeding under the first, soft pad as the first, soft pad rests on the skin of the patient.

21. The method of claim 17 in which the first pad is thin enough to allow palpitation of a blood vessel of the patient through the pad, and including the step of palpitating the blood vessel through the pad prior to passing the medical cannula through the first pad and into the blood vessel.

22. The method of claim 1 in which the first pad is thin enough to allow palpitation of a blood vessel of the patient through the pad, and including the step of palpitating the blood vessel through the pad prior to passing the medical cannula through the first pad and into the blood vessel.
